(19) United States
(12) Patent Application Publication
(10) Pub. No.: US 2023/0101109 A1
(43) Pub. Date: Mar. 30, 2023

(54) STAPLING DEVICE HAVING ACTUATION SLED WITH KNIFE GUARD

(71) Applicant: Covidien LP, Mansfield, MA (US)

(72) Inventor: Justin P. Williams, Southbury, CT (US)

(21) Appl. No.: 17/488,639

(22) Filed: Sep. 29, 2021

(51) Int. Cl. A61B 17/072

(52) U.S. Cl. CPC A61B 17/07207; A61B 2017/07278; A61B 2017/07271; A61B 2017/07285; A61B 2017/00367

(57) ABSTRACT

A surgical stapling device includes a tool assembly having an anvil assembly, a cartridge assembly, and a drive assembly. The cartridge assembly includes a channel member that defines a recess and a staple cartridge that is releasably received within the recess of the channel member. The staple cartridge includes a cartridge body, staples, pushers, and an actuation sled that supports a knife. The drive assembly has a working member that is movable through the tool assembly to advance the actuation sled through the staple cartridge to actuate the tool assembly. The actuation sled of the staple cartridge includes a guard to shield the knife and minimize any likelihood of injury to medical personnel during installation and replacement of the staple cartridge.

### FIELD

The disclosure is directed to surgical stapling devices and, more particularly, to surgical stapling devices having actuation sleds that support knives.

### BACKGROUND

Surgical stapling devices for simultaneously stapling and cutting tissue are well known in the art and include a tool assembly and a drive assembly. The tool assembly includes an anvil assembly and a cartridge assembly that are movable in relation to each other between open and clamped positions. The cartridge assembly has a channel member and a staple cartridge that is received within the channel member and is replaceable to facilitate reuse of the stapling device. The staple cartridge includes a cartridge body that supports staples, pushers, and an actuation sled. The drive assembly is positioned proximally of the actuation sled and is movable in relation to the anvil and cartridge assemblies to advance the actuation sled from its retracted position to its advanced position to eject the staples from the cartridge body. Typically, the actuation sled remains in its advanced position when the drive assembly is returned to its retracted position after firing of the stapling device.

In some stapling devices, a knife is supported on the actuation sled and is movable with the actuation sled through the cartridge body to cut tissue clamped between the anvil and cartridge assemblies. When the drive assembly is retracted and the actuation sled and the knife remain in a distal portion of the cartridge body, exposure of the knife can be a hazardous to medical personnel.

A continuing need exists for in the art for a staple cartridge that is configured to minimize any likelihood of injury to medical personnel during installation and/or replacement of the staple cartridge.

### SUMMARY

This disclosure is directed to a surgical stapling device that includes a tool assembly having an anvil assembly, and a cartridge assembly that has a channel member and a staple cartridge. The staple cartridge includes a cartridge body, staples, pushers, and an actuation sled that supports a knife. The drive assembly has a working member that is movable through the tool assembly to advance the actuation sled through the staple cartridge to actuate the tool assembly. The actuation sled of the staple cartridge includes a guard to shield the knife to minimize any likelihood of injury to medical personnel during installation and replacement of the staple cartridge.

Aspects of this disclosure are directed to a surgical stapling device including an elongate body and a tool assembly. The elongate body has a proximal portion and a distal portion. The tool assembly is supported on the distal portion of the elongate body and includes a cartridge assembly and an anvil assembly. The cartridge assembly has a channel member that defines a recess and a staple cartridge that is received within the recess. The staple cartridge includes a cartridge body, an actuation sled, pushers, and staples. The cartridge body defines a central knife slot and staple receiving pockets positioned on opposite side of the central knife slot. Each of the staple receiving pockets receives one of the pushers and one of the staples. The actuation sled includes a central portion, a cam member positioned on each side of the central portion, a vertical portion, and a knife guard. The vertical portion extends upwardly from the central portion and supports a knife. The knife guard extends distally from the vertical portion and is positioned above the knife such that the knife is confined between the central portion and the knife guard. The actuation sled is movable from a retracted position to an advanced position to eject the staples from staple receiving slots of the cartridge body. The anvil assembly is coupled to the cartridge assembly to facilitate movement of the tool assembly between open and clamped positions. The drive assembly is positioned proximally of the actuation sled and is movable between a retracted position and an advanced position to move the actuation sled from its retracted position to its advanced position.

In aspects of the disclosure, the knife guard extends distally from the central portion of the actuation sled to a position above the cam members of the actuation sled.

In aspects of the disclosure, the actuation sled is of monolithic construction.

In some aspects of the disclosure, the knife guard and the vertical portion of the actuation sled are fixedly secured to the central portion of the actuation sled.

In certain aspects of the disclosure, the vertical portion of the actuation sled is positioned within the central knife slot of the cartridge body and extends above the cartridge body.

In aspects of the disclosure, the cartridge body includes protrusions, and the central portion of the actuation sled defines concavities that receive the protrusions when the actuation sled is in its retracted position to releasably retain the actuation sled in its retracted position.

In some aspects of the disclosure, the knife is secured to the vertical portion of the actuation sled by over molding.

In certain aspects of the disclosure, the stapling device includes a handle assembly that is secured to the proximal portion of the elongate body.

In aspects of the disclosure, the vertical portion of the actuation sled is secured to a proximal portion of the central portion of the actuation sled.

In some aspects of the disclosure, the drive assembly includes a flexible drive beam and a working member.

In certain aspects of the disclosure, the flexible drive beam has a proximal portion and a distal portion, and the working member is supported on the distal portion of the flexible drive beam.

In aspects of the disclosure, the working portion includes a first beam, a second beam, and a vertical strut connecting the first beam to the second beam.

In some aspects of the disclosure, the drive assembly is movable from a retracted position to an advanced position to move the actuation sled from its retracted position to its advanced position.

In certain aspects of the disclosure, the anvil assembly defines a channel that extends longitudinally along the anvil assembly.

In aspects of the disclosure, the first beam is movable through channel of the anvil assembly as the drive assembly is moved from its retracted position to its advanced position.

Other aspects of the disclosure are directed to a staple cartridge including a cartridge body, an actuation sled, pushers, and staples. The cartridge body defines a central knife slot and staple receiving pockets that are positioned on opposite side of the central knife slot. Each of the staple receiving pockets receives one of the pushers and one of the staples. The actuation sled includes a central portion, a cam member positioned on each side of the central portion, a vertical portion, and a knife guard. The vertical portion extends upwardly from the central portion and supports a knife. The knife guard extends distally from the vertical portion and is positioned above the knife such that the knife is confined between the central portion and the knife guard. The actuation sled is movable from a retracted position to an advanced position to eject the staples from staple receiving slots of the cartridge body.

Still other aspects of the disclosure are directed to an actuation sled including a central portion, a cam member positioned on each side of the central portion, a vertical portion, and a knife guard. The vertical portion extends upwardly from the central portion and supports a knife. The knife guard extends distally from the vertical portion and is positioned above the knife such that the knife is confined between the central portion and the knife guard. The actuation sled is movable from a retracted position to an advanced position to eject the staples from staple receiving slots of the cartridge body.

Other features of the disclosure will be appreciated from the following description.

### BRIEF DESCRIPTION OF THE DRAWINGS

Various aspects of the disclosure are described herein below with reference to the drawings, wherein:

FIG. 1 is side perspective view of a stapling device according to aspects of the disclosure including a tool assembly in an open position;

FIG. 2 is an enlarged view of the indicated area of detail shown in FIG. 1 illustrating the tool assembly of the stapling device shown in FIG. 1 with the anvil assembly shown in phantom;

FIG. 3 is a side perspective view of the tool assembly shown in FIG. 2 with a staple cartridge of a cartridge assembly of the stapling device removed from a channel member of the staple cartridge;

FIG. 4 is an exploded perspective view of the staple cartridge shown in FIG. 3;

FIG. 5 is an enlarged view of the indicated area of detail shown in FIG. 4;

FIG. 6 is an enlarged view of the indicated area of detail shown in FIG. 3;

FIG. 7 is a side perspective view of a distal portion of a drive assembly of the stapling device shown in FIG. 1;

FIG. 8 is a side perspective view of the tool assembly shown in FIG. 2 in a clamped position about tissue with the tissue shown in phantom;

FIG. 9 is a cross-sectional view taken along section line 9-9 of FIG. 8; and

FIG. 10 is a side perspective view of the tool assembly shown in FIG. 8 in the open position after the stapling device has been fired.

### DETAILED DESCRIPTION

The disclosed surgical stapling device will now be described in detail with reference to the drawings in which like reference numerals designate identical or corresponding elements in each of the several views. However, it is to be understood that aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the stapling device in its customary manner, while the term “distal” is used generally to refer to that portion of the device that is farther from the clinician during use of the stapling device in its customary manner. In addition, the term “clinician” is used generally to refer to medical personnel including doctors, nurses, and support personnel. Further, directional terms such as front, rear, upper, lower, top, bottom, and similar terms are used to assist in understanding the description and are not intended to limit the disclosure.

The disclosed surgical stapling device includes a tool assembly having an anvil assembly, a cartridge assembly, and a drive assembly. The cartridge assembly includes a channel member that defines a recess and a staple cartridge that is releasably received within the recess of the channel member. The staple cartridge includes a cartridge body, staples, pushers, and an actuation sled that supports a knife. The drive assembly has a working member that is movable through the tool assembly to advance the actuation sled through the staple cartridge to actuate the tool assembly. The actuation sled of the staple cartridge includes a knife guard to shield the knife and minimize any likelihood of injury to medical personnel during installation and replacement of the staple cartridge.

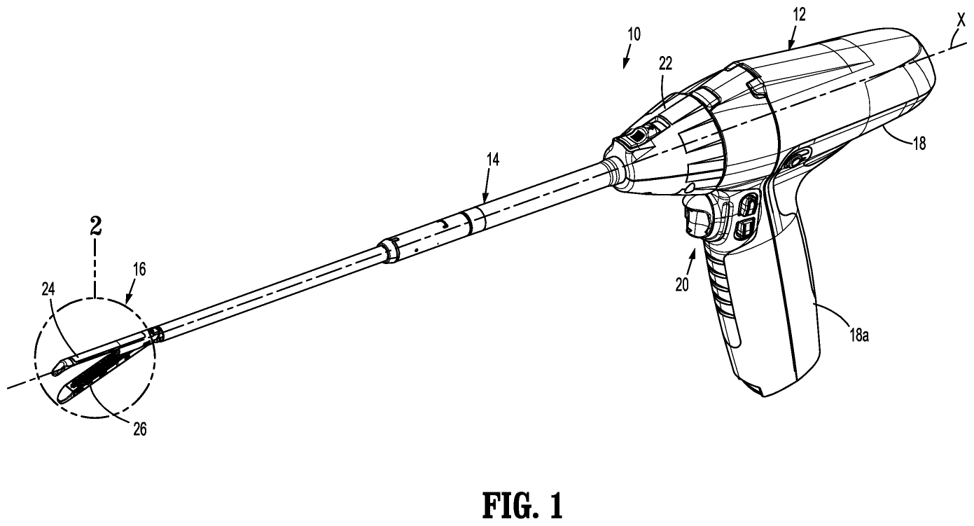

FIG. 1 illustrates a surgical stapling device according to aspects of the disclosure shown generally as stapling device 10. The stapling device 10 includes a handle assembly 12, an elongate body 14, and a tool assembly 16. The elongate body 14 defines a longitudinal axis “X” (FIG. 1). The handle assembly 12 includes a body 18 that defines a hand grip 18a, a plurality of actuator buttons 20, and a rotation knob 22. The rotation knob 22 is rotatably supported on a distal portion of the body 18 of the handle assembly 12 and supports the elongate body 14 to facilitate rotation of the elongate body 14 and the tool assembly 16 about the longitudinal axis “X” of the elongate body 14 in relation to the handle assembly 12. The actuator buttons 20 control operation of the various functions of the stapling device 10 including articulation, clamping, firing, and cutting of tissue.

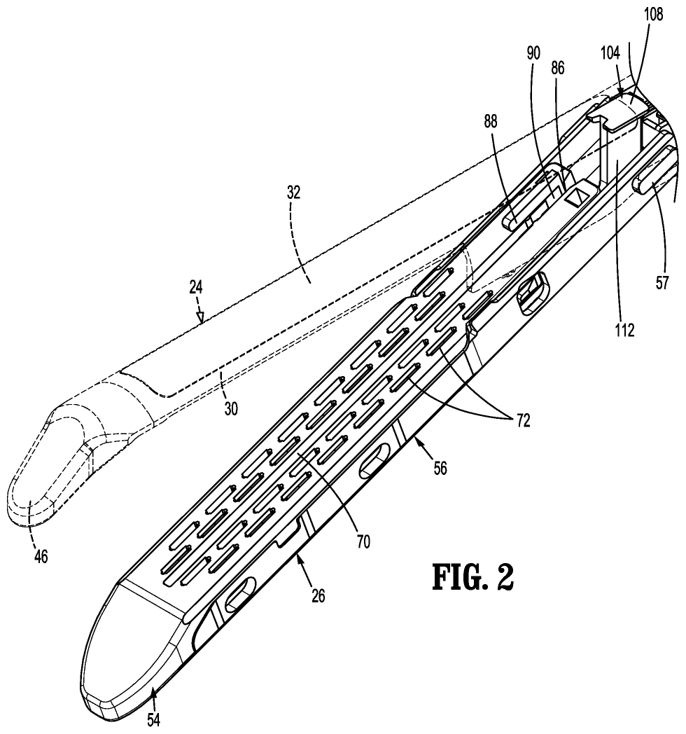

FIG. 2 illustrates the tool assembly 16 which includes an anvil assembly 24 and a cartridge assembly 26 that are movable in relation to each other between open and clamped positions. As illustrated, the anvil assembly 24 is fixed in relation to the elongate body 14 and the cartridge assembly 26 is pivotable towards the anvil assembly 24. However, it is envisioned that the cartridge assembly 26 could be fixed in relation to the elongate body 14 and the anvil assembly 24 could pivot towards the cartridge assembly 26. Although the stapling device 10 is illustrated as an electrically powered stapling device, it is envisioned that the tool assembly 16 would also be suitable for use with manually powered surgical stapling devices and robotically operated stapling systems. U.S. Pat. No. 9,055,943 discloses a surgical stapling device including a powered handle assembly, U.S. Pat. No. 6,241,139 discloses a surgical stapling device including a manually actuated handle assembly, and U.S. Pat. No. 9,962,159 discloses a stapling device that is configured for use with a robotic system.

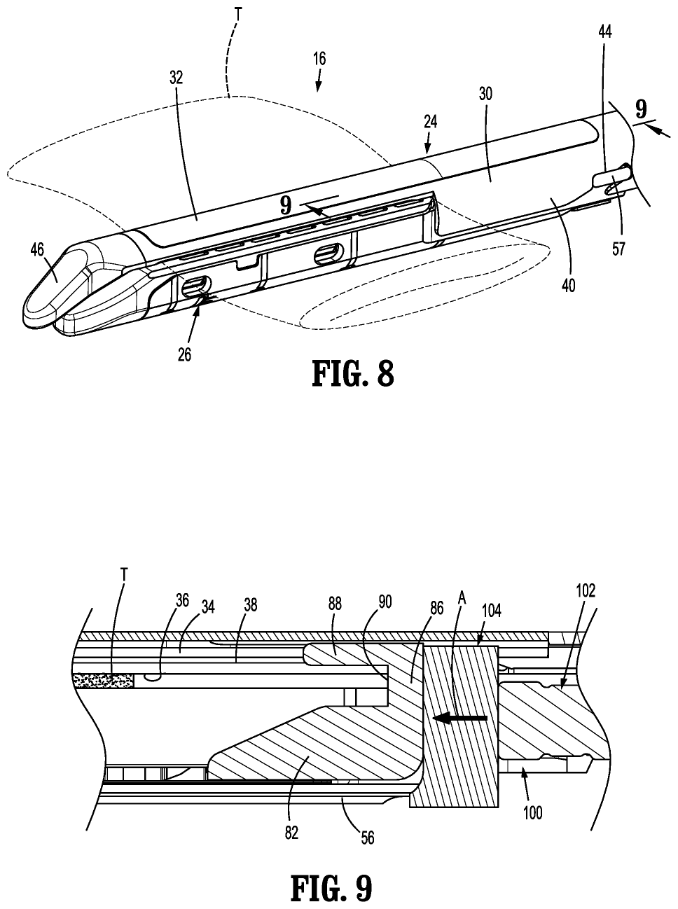

The anvil assembly 24 includes an anvil portion 30 and a cover portion 32 that is attached to the anvil portion 30 to define a channel 34 (FIG. 8) within the anvil assembly 24. The anvil portion 30 includes a staple forming surface 36 (FIG. 8) that defines a central knife slot 38 (FIG. 8) that extends along a longitudinal axis of the anvil portion 30. The anvil portion 30 includes a mounting portion 40 that includes extensions 42 that extend towards the cartridge assembly 26. Each of the extensions defines a cutout 44. In some aspects of the disclosure, the anvil portion 30 of the anvil assembly 24 supports a tissue dissector 46.

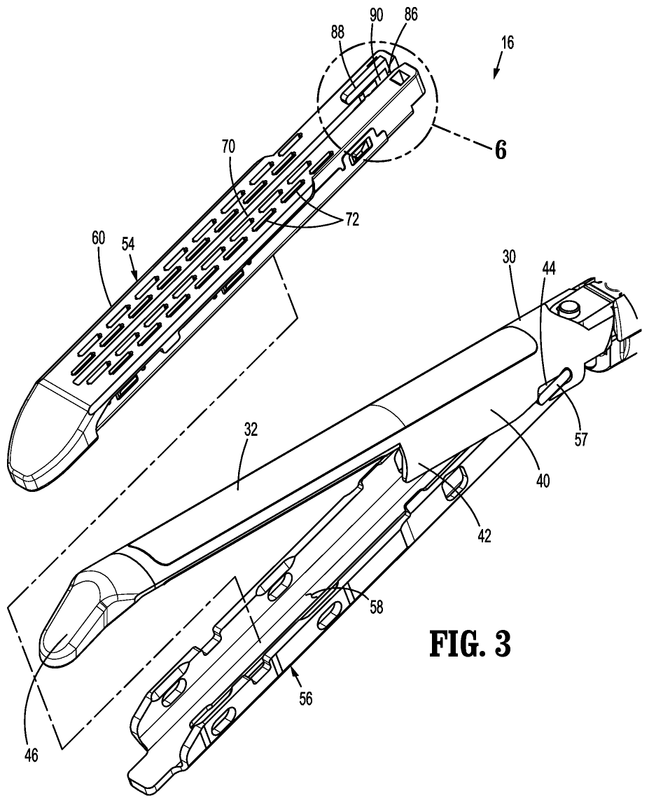

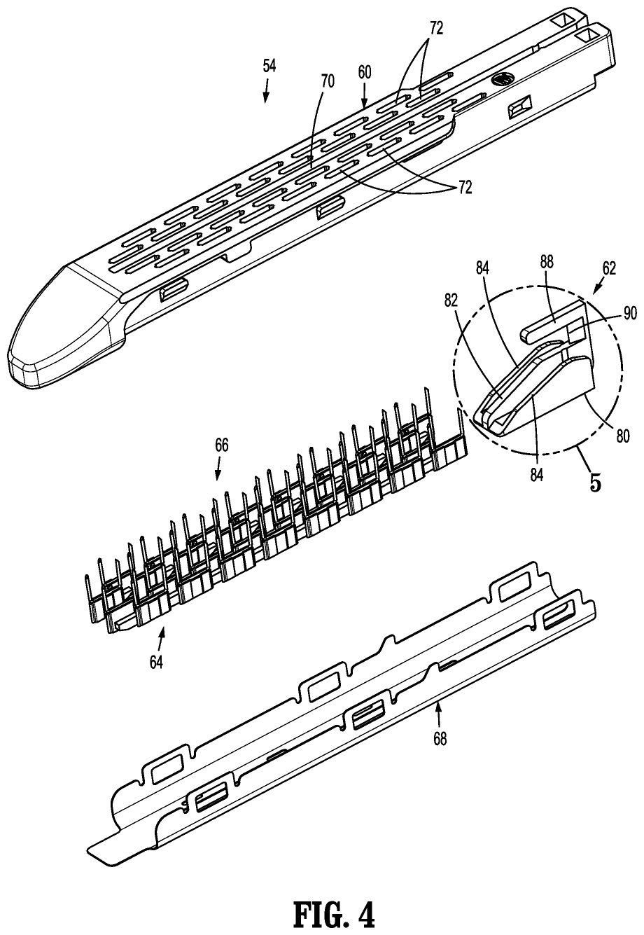

FIGS. 2-4 illustrate the cartridge assembly 26 which includes a staple cartridge 54 and a channel member 56 that defines a recess 58 that receives the staple cartridge 54. In aspects of the disclosure, the staple cartridge 54 is releasably received within the recess 58 of the channel member 56 and is replaceable to facilitate reuse of the stapling device 10. The channel member 56 includes a pivot member 57 that extends outwardly from side walls of the channel member 56 and is received in the cutout 44 (FIG. 3) of the anvil assembly 24 to pivotably couple the cartridge assembly 26 to the anvil assembly 24. In aspects of the disclosure, the cartridge assembly 26 can pivot in relation to the anvil assembly 24 between the open and clamped positions.

The staple cartridge 54 includes a cartridge body 60, an actuation sled 62, pushers 64, staples 66, and a base plate 68. The cartridge body 60 defines a central knife slot 70 and staple receiving pockets 72 and includes protrusions 74 (FIG. 6) that extend into the central knife slot 70. In aspects of the disclosure, the staple receiving pockets 72 are aligned in rows that are positioned on each side of the central knife slot 70. Although two rows of staple receiving pockets 72 are shown on each side of the central knife slot 70, it is envisioned that the cartridge body 60 may define one or more rows of staple receiving pockets 72 on each side of the central knife slot 70, e.g., three. Each of the staple receiving pockets 72 receives one of the staples 66 and one of the pushers 64. The pushers 64 support the staples 66 and are engaged by the actuation sled 62 as the actuation sled 62 is advanced through the cartridge body 60 from a retracted position to an advanced position to eject the staples 66 from the cartridge body 60. The base plate 68 is secured to the bottom of the cartridge body 60 to prevent the pushers 64 and the staples 66 from falling from the cartridge body 60.

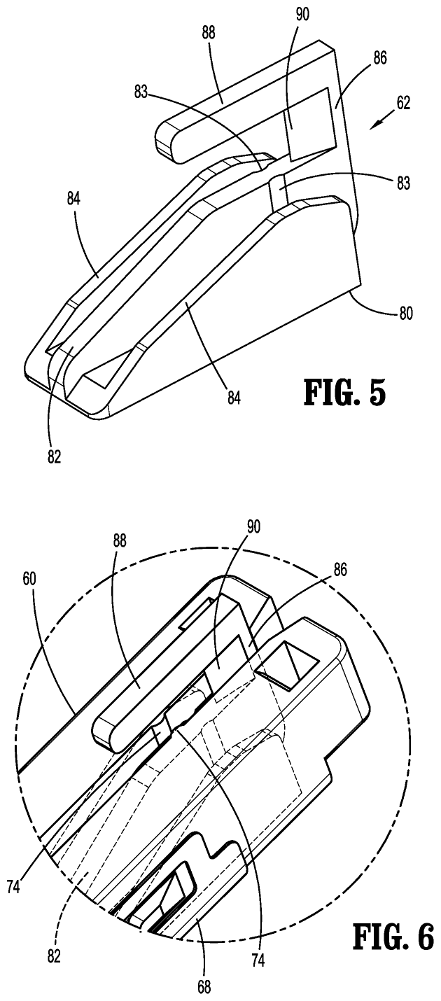

FIGS. 5 and 6 illustrate the actuation sled 62 which includes a body 80 that has a central portion 82 and cam surfaces 84 positioned on opposite sides of the central portion 82 of the body 80. The central portion 82 is received within the central knife slot 70 of the cartridge body 60 and defines radiused concavities 83. The radiused concavities 83 receive the protrusions 74 of the cartridge body 60 when the actuation sled 62 is in a retracted position to releasably retain the actuation sled 70 in its retracted position. The cam surfaces 84 define ramps that are positioned to engage the pushers 64 as the actuation sled 62 moves from its retracted position towards its advanced position to sequentially eject the staples 66 from the cartridge body 60. The central portion 82 of the body 80 of the actuation sled 62 includes a vertical portion 86 and a knife guard 88. The vertical portion 86 of the actuation sled 62 extends upwardly from a proximal portion of the central portion 82 of the actuation sled 62 through the central knife slot 70 in the cartridge body 60 (FIG. 6) and includes a knife 90 that faces distally. In aspects of the disclosure, the knife 90 is formed of metal and is over molded to the vertical portion of the actuation sled 62. Alternately, the knife 90 can be machined into the vertical portion 86 of the actuation sled 62.

The knife guard 88 extends distally from the vertical portion 86 of the actuation sled 62 over the knife 90 along a longitudinal axis of the tool assembly 16 to guard or shield the knife 90. In aspects of the disclosure, the knife guard 88 extends along an axis that is substantially parallel to the longitudinal axis of the tool assembly 16. In aspects of the disclosure, the knife guard 88 extends distally from the proximal portion of the central portion 82 of the actuation sled 62 to a position above the cam surfaces 84 of the actuation sled 62 and is fixed in relation to the central portion 82 of the actuation sled 62 such that the knife 90 is positioned or confined between the central portion 86 of the actuation sled 62 and the knife guard 88. In some aspects of the disclosure, the actuation sled 62 is molded or formed as a monolithic structure. Alternately, the vertical portion 86 and/or the knife guard 88 of the actuation sled 62 can be secured to the body 80 of the actuation sled using a variety of securement techniques, e.g., over molding.

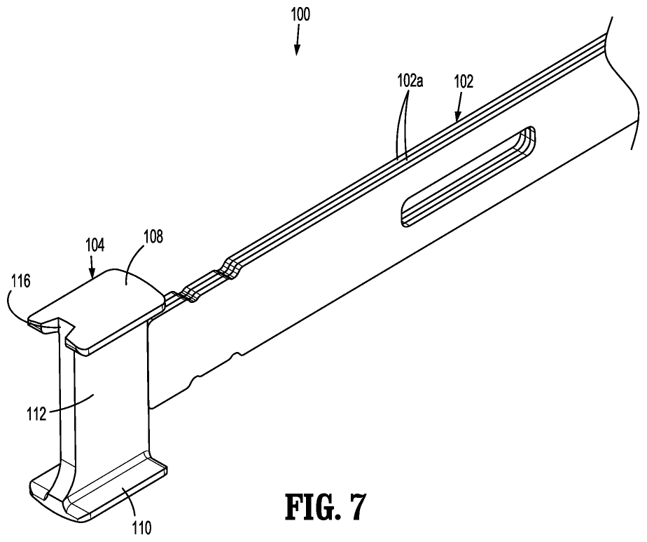

FIG. 7 illustrates a drive assembly 100 of the stapling device 10 (FIG. 1). The drive assembly 100 includes a resilient and/or flexible drive beam 102 and a working member 104. The drive beam 102 has a proximal portion (not shown) that is coupled to a drive rod (not shown) supported within the elongate body 14 of the stapling device 10 (FIG. 1) and a distal portion that is coupled to the working member 104. In aspects of the disclosure, the resilient drive beam 102 is formed from laminated sheets 102a of material, e.g., steel, that are welded to the working member 104. Alternately other materials of construction and securement techniques are envisioned.

The working member 104 of the drive assembly 100 has an I-beam configuration and includes a first beam 108, a second beam 110, and a vertical strut 112 that connects the first beam 108 to the second beam 110. The working member 104 is positioned proximally of the actuation sled 62 (FIG. 9) and is movable, in response to activation of the actuation buttons 20 (FIG. 1), through the tool assembly 16 between retracted and advanced positions to move the actuation sled 62 through the tool assembly 16 (FIG. 1) from its retracted position to its advanced position. As the working member 104 moves through the cartridge assembly 26, the first beam 108 is received within the channel 34 (FIG. 9) defined within the anvil assembly 24, the second beam 110 is engaged with the cartridge assembly 26, and the vertical strut 112 moves through the central knife slots 38 (FIG. 3) and 70 (FIG. 6) of the anvil portion 36 and cartridge body 60, respectively. The first beam 108 of the working member 104 extends distally of the vertical strut 112 to form an overhang that defines a cutout 116.

FIGS. 8 and 9 illustrate the tool assembly 16 of the stapling device 10 (FIG. 1) in the clamped position. When the drive assembly 100 is moved in the direction of arrow ‘A” from its retracted position towards the advanced position, the second beam 110 of the working member 104 of the drive assembly 100 engages a cam surface (not shown) of the channel member 56 of the cartridge assembly 26 to pivot the cartridge assembly 26 upwardly towards the anvil assembly 24 in the direction of arrow “B” to move the tool assembly 16 from the open position to the clamped position about tissue “T”. As can be seen, in the clamped position, the working member 104 of the vertical strut 112 of the drive assembly 100 is engaged with, or in close approximation to, a proximal surface of the actuation sled 62. Upon continued advancement of the drive assembly 100 in the direction of arrow “A”, the actuation sled 62 advances through the cartridge body 60 of the staple cartridge 54 to eject the staples 66 from the staple receiving pockets 72 through the tissue “T” into the staple forming surface 36 of the anvil assembly 24 to form the staples 66 in the tissue “T”.

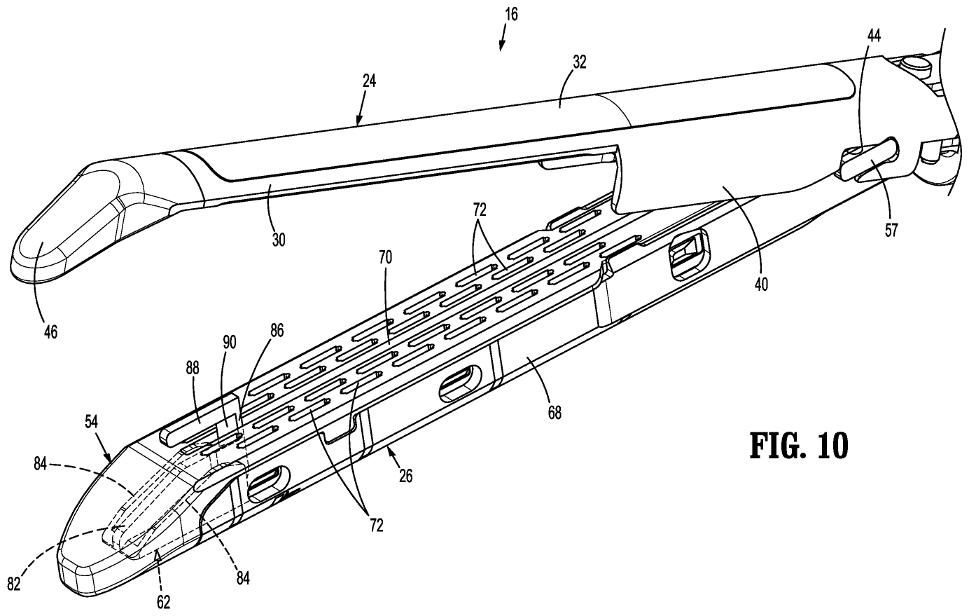

FIG. 10 illustrates the tool assembly 16 of the stapling device 10 (FIG. 1) after the stapling device 10 has been fired and the drive assembly 100 is returned to its retracted position. As illustrated, the knife 90 extends from the central knife slot 70 near a distal end of the of the cartridge body 60 and is shielded by the knife guard 88 of the actuation sled 62. As described above, the knife guard 88 of the actuation sled 62 is fixed to the body 80 of the actuation sled 62 and shields the knife 90 in both the retracted position (FIG. 2) and the advanced position (FIG. 10) of the actuation sled 62 to protect a clinician from inadvertent injury during installation and removal of the staple cartridge 54 from the channel member 56 of the cartridge assembly 26.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A surgical stapling device comprising:
an elongate body having a proximal portion and a distal portion;
a tool assembly supported on the distal portion of the elongate body, the tool assembly including:
a cartridge assembly having a channel member and a staple cartridge, the channel member defining a recess that receives the staple cartridge, the staple cartridge including a cartridge body, an actuation sled, pushers, and staples, the cartridge body defining a central knife slot and staple receiving pockets positioned on opposite side of the central knife slot, each of the staple receiving pockets receiving one of the pushers and one of the staples, the actuation sled including a central portion, a cam member positioned on each side of the central portion, a vertical portion, and a knife guard, the vertical portion extending upwardly from the central portion and supporting a knife, the knife guard extending distally from the vertical portion and being positioned above the knife such that the knife is confined between the central portion and the knife guard, the actuation sled movable from a retracted position to an advanced position to eject the staples from staple receiving slots of the cartridge body; and
an anvil assembly coupled to the cartridge assembly to facilitate movement of the tool assembly between open and clamped positions; and
a drive assembly positioned proximally of the actuation sled and being movable between a retracted position and an advanced position to move the actuation sled from its retracted position to its advanced position.

2. The stapling device of claim 1, wherein the knife guard extends distally from the central portion of the actuation sled to a position above the cam members of the actuation sled.

3. The stapling device of claim 1, wherein the actuation sled is of monolithic construction.

4. The stapling device of claim 1, wherein the knife guard and the vertical portion of the actuation sled are fixedly secured to the central portion of the actuation sled.

5. The stapling device of claim 1, wherein the vertical portion of the actuation sled is positioned within the central knife slot of the cartridge body and extends above the cartridge body.

6. The stapling device of claim 1, wherein the cartridge body includes protrusions, and the central portion of the actuation sled defines concavities that receive the protrusions when the actuation sled is in its retracted position to releasably retain the actuation sled in its retracted position.

7. The stapling device of claim 1, wherein the knife is secured to the vertical portion of the actuation sled by over molding.

8. The stapling device of claim 1, further including a handle assembly secured to the proximal portion of the elongate body.

9. The stapling device of claim 1, wherein the vertical portion of the actuation sled is secured to a proximal portion of the central portion of the actuation sled.

10. The stapling device of claim 1, wherein the drive assembly includes a flexible drive beam and a working member, the flexible drive beam having a proximal portion and a distal portion, the working member supported on the distal portion of the flexible drive beam and including a first beam, a second beam, and a vertical strut connecting the first beam to the second beam, the drive assembly movable from a retracted position to an advanced position to move the actuation sled from its retracted position to its advanced position.

11. The stapling device of claim 10, wherein the anvil assembly defines a channel that extends longitudinally along the anvil assembly.

12. The stapling device of claim 11, wherein the first beam is movable through channel of the anvil assembly as the drive assembly is moved from its retracted position to its advanced position.

13. A staple cartridge comprising:
a cartridge body, an actuation sled, pushers, and staples, the cartridge body defining a central knife slot and staple receiving pockets positioned on opposite side of the central knife slot, each of the staple receiving pockets receiving one of the pushers and one of the staples, the actuation sled including a central portion, a cam member positioned on each side of the central portion, a vertical portion, and a knife guard, the vertical portion extending upwardly from the central portion and supporting a knife, the knife guard extending distally from the vertical portion and being positioned above the knife such that the knife is confined between the central portion and the knife guard, the actuation sled movable from a retracted position to an advanced position to eject the staples from staple receiving slots of the cartridge body.

14. The staple cartridge of claim 13, wherein the knife guard extends distally from the central portion of the actuation sled to a position above the cam members of the actuation sled.

15. The staple cartridge of claim 13, wherein the knife guard and the vertical portion of the actuation sled are fixedly secured to the central portion of the actuation sled.

16. The staple cartridge of claim 13, wherein the vertical portion of the actuation sled is positioned within the central knife slot of the cartridge body and extends above the cartridge body.

17. The staple cartridge of claim 13, wherein the cartridge body includes protrusions, and the central portion of the actuation sled defines concavities that receive the protrusions when the actuation sled is in its retracted position to releasably retain the actuation sled in its retracted position.

18. The staple cartridge of claim 13, wherein the knife is secured to the vertical portion of the actuation sled by over molding.

19. The staple cartridge of claim 13, wherein the vertical portion of the actuation sled is secured to a proximal portion of the central portion of the actuation sled.

20. An actuation sled comprising:
a central portion, a cam member positioned on each side of the central portion, a vertical portion, and a knife guard, the vertical portion extending upwardly from the central portion and supporting a knife, the knife guard extending distally from the vertical portion and being positioned above the knife such that the knife is confined between the central portion and the knife guard, the actuation sled movable from a retracted position to an advanced position to eject the staples from staple receiving slots of the cartridge body.
